(19) United States
(12) Patent Application Publication
(10) Pub. No.: US 2020/0330790 A1
(43) Pub. Date: Oct. 22, 2020

(54) Laser Device with Gem Stone for Holistic Therapy

(71) Applicant: Yat Fai Wong, Vancouver (CA)

(72) Inventor: Yat Fai Wong, Vancouver (CA)

(21) Appl. No.: 16/387,574

(22) Filed: Apr. 18, 2019

(51) Int. Cl. A61N 5/06; H01S 5/022

(52) U.S. Cl. CPC A61N 5/0619; A61N 2005/067; H01S 5/0228

(57) ABSTRACT

A laser device with gem stone for holistic therapy is disclosed. The device being comprised of three main parts: a first part being a pen-type, handheld, low-level laser, a second part being a gem stone positioned to an end of said laser; and a terminal sleeve portion fixedly-connecting the aforementioned gem stone to the laser. Unlike existing holistic therapy laser devices, the current disclosure allows the gem stone to refract the laser into several beams thereby treating multiple points on the body simultaneously. Another embodiment of the invention is comprised of a gem stone cap configured to be retrofitted onto existing, conventional laser pointers and being removable to allow a user to install a variety of gem stones therein.

### FIELD OF THE INVENTION

The present invention relates to a laser device. More specifically, the present invention relates to a laser device with a gem tip for holistic therapies.

### BACKGROUND OF THE INVENTION

Holistic therapies such as chakra balancing and acupuncture have been in existence for centuries. The chakra system originated in India between 1500 and 0.500 B.C. in the oldest text called the Vedas. Evidence of chakras is also found in the Shri Jabala Darshana Upanishad, the Cudamini Upanishad, the Yoga-Shikka Upanishad and the Shandilya Upanishad documents. The word Chakra is defined as a spinning disk or wheel of energy that runs along the spine. There are seven ‘main’ chakras along the spine, though some say there are as many as 114 all over the body. The health of one's chakras is considered to be directly connected to the health of the physical body, the mind, and the emotional wellbeing of a person. Acupuncture is an ancient system of healing that began during the Stone Age when sharp edged tools and stones were used to puncture and drain abscesses. The basis of modern acupuncture was established during the Ming Dynasty in the 1300s with the publication of The Great Compendium of Acupuncture and Moxibustion. Previously, during the sixth century, Traditional Chinese Medicine and acupuncture were introduced into Japan via improved transportation. Acupuncture has been used in the West since the 17th century and was first recorded in Europe during the 1800s. Acupuncture became increasingly popular in the United States after President Nixon's visit to China in 1972 and learned about acupuncture. The U.S. Food and Drug Administration classified acupuncture needles as medical instruments in 1995.

Laser acupuncture therapy has hem used as an alternative or complementary treatment of traditional acupuncture therapy. In a laser acupuncture therapy, a low power laser beam is used to replace a needle to stimulate an acupuncture point on a patients body. Such treatment is less painful (compared to traditional needle treatment. Various laser devices have been developed and used for acupuncture treatment based on such lasers as He—Ne or diode lasers. These Laser acupuncture devices implement one or two laser output channels delivered from a hand-held applicator. To use these devices, a therapist needs to hold the applicator steadily to point the laser beam onto an acupuncture point. In an acupuncture treatment, stimulating five to ten acupuncture points are common and each point takes typically rive to thirty minutes. Thus, a therapist needs to point the laser beam to one acupuncture point then another for a long time. Therapists have only recently begun using translucent, stones in combination with tight energy. United States Patent No. US20140271556A1 disclosed a device that shines visible light through crystals to treat patients. Japanese and German Patent Nos. JP2006006677A and DE19547106A1 disclosed devices that also shine visible light through gem stones as a form of holistic therapy. While many of these devices use visible light, none were found that combined laser light with gem stones to refract light into a plurality of rays for acupuncture and chakra therapy.

### SUMMARY OF THE INVENTION

A laser device with gem stone for holistic therapy is disclosed. An object of the invention is to independently activate energy points on the body. For example, a user can shine the device on acupuncture and chakra points manually without the need for a practitioner.

Another object of the invention is to stimulate energy points on the body without discomfort. For example, a user can shine the device on acupuncture and chakra points manually without the discomfort that usually occurs with traditional needling.

Another object of the invention is to treat multiple energy points on the body with a single, device. For example, in traditional acupuncture treatments, multiple needles are inserted into multiple points to treat an ailment. In the current disclosure, the laser is split (or refracted) into multiple beams as it passes through a gem stone, allowing a user to treat multiple points with a single device. As the device is moved away from the skin, the beams widen. As the device is moved closer to the skin the beams become mote narrowed and focused.

Another object of the invention is to allow users to select from an infinite number of gem stone types and stone shapes in order to produce an infinite number of laser treatment patterns. For example a user can switch between a sapphire and a gem simply by removing the cap on the end of the device.

Another object of the invention Ls to allow users to easily transport a form of light treatment with diem. For example, a user can place the device in a shirt pocket and remove it easily for on-the-spot treatments. Conventional therapies are often too large to transport, easily.

Another object of the invention is to provide a device with an onboard power source. The invention runs on conventional pen-light batteries drat are much smaller than similar devices that need to be plugged into a wall outlet in order to function.

### BRIEF DESCRIPTION OF THE DRAWINGS

FIG. 1 is a perspective view or the device.

FIG. 2 is a section new of the device.

FIG. 3 is a side view of the device.

FIG. 4 is a perspective view of a cap retrofit embodiment of the invention.

### DETAILED DESCRIPTION OF THE PREFERRED EMBODIMENTS

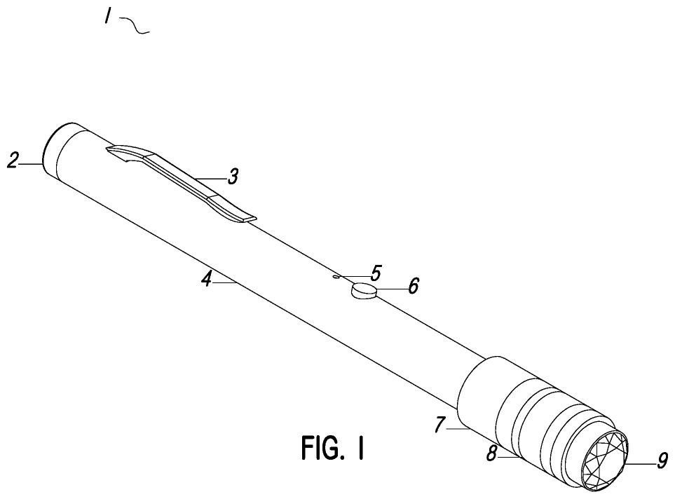

FIG. 1 shows a perspective view an embodiment of the invention t being comprised of a hollow, barrel 4 and pocket dip 3 on an end both parts being made of a rigid material including but not limited to metal, plastic and the like. One distal end of the device having a selectively removable, threaded end cap 2. Another distal end of the device having head 7 made of a precious metal such as, but not limited to silver, bronze etc. and disposed to receive a gem stone 9 therein. Said gem stone 9 being comprised of a gem such as but not limited to sapphire, emerald etc. and being selectively affixed to the head 7. A power button 6 with LED status light 5 being disposed proximally on said barrel 4.

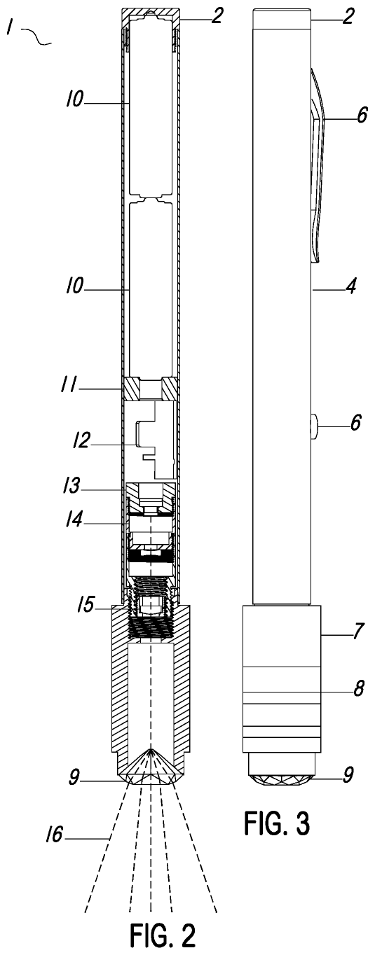

FIG. 2 allowing a section view of an embodiment of invention 1 with at least two AA-sized batteries 10 therein being selectively held in place by means of end cap 2 and insulation plate 11 there between. Other haltenes may be utilized such as but not limited to rechargeable, nickel hydrido, lithium ion and the like and providing electrical power to laser module 13. Use power output laser power from the diode laser module 13 should be about, but not limited to 5 mW, while a range of 1 mW to 100 mW may be used. The emitting wavelength of the diode laser module 13 is selected to have a desirable penetration depth for effectively stimulating an acupuncture point. For example, the laser wavelength may be at or around 635 nm to match the He—Ne laser or near 830 nm to increase the laser penetration depth.

Any wavelength ranged from 500 nm to 1500 nm may be chosen for a variety of laser acupuncture treatments. The figure showing laser light moving through lens barrel 15 and laser hole 15 and entering gem stone 9. Said gem stone 9 refracting and splitting laser light into a plurality of beams 16 therefrom.

FIG. 3 showing a side view of an embodiment of the invention 1 with the aforementioned head 7 having a plurality of slots 8 being arranged in a ‘Fibonacci,’ proportional pattern thereon.

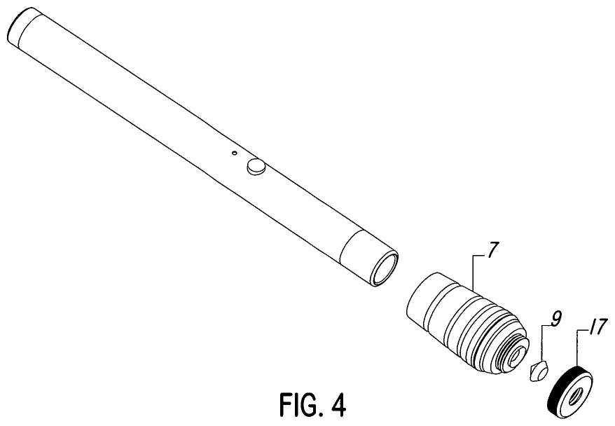

FIG. 4 showing a perspective view of a cap retrofit embodiment of the invention. This other embodiment being comprised of head 17 also made of a rigid material including but not limited to 3D printed plastics (nylon, PLA, SLA, polycarbonate etc.) silver, bronze and the like and breaded end cap 17 configured to receive a gem stone 9 there between. Said end cap 17 having a knurling relief on an exterior portion with an annular space disposed proximally. The inner portion of said annular space being configured to allow gem stone 9 to be mounted similar to that of conventional jewelry. Said end cap 17 being selectively removed from the head 17 to allow the replacement of various gem stones therein. The cap retrofit embodiment being configured to receive conventional laser pointing devices therein. Finally, the aforementioned head 7 having a plurality of slots 8 being arranged in a ‘Fibonacci,’ proportional pattern thereon.

What is claimed is:

1. A laser apparatus for laser acupuncture, and chakra therapy, comprising
(a) a diode laser;
(b) a plurality of batteries;
(c) a power switch;
(d) a pocket clip;
(e) a status LED;
(f) a head; and
(g) a gem stone.

2. The diode laser of claim 1 producing a laser beam at a selected wavelength that has a desired penetration depth into a patient's body part.

3. The head of claim 1 configured to hold a gem stone there in.

4. The head of claim 1 configured to allow a user to exchange gem stones there in.

5. The head of claim 1 having an embodiment that is retrofitted onto existing laser pointers.

6. The diode laser in claim 1 wherein said selected wavelength is around 635 nm or 830 nm.

7. The diode laser in claim 1 wherein said output power of said diode laser modules is approximately 5 mW.

8. A method of activating acupuncture and chakra points. The method comprising:
(a) delivering light energy;

9. The light energy in claim 7 further including a splitting up of the light into multiple beams.
